(19) United States
(12) Patent Application Publication
(10) Pub. No.: US 2022/0233797 A1
(43) Pub. Date: Jul. 28, 2022

(54) METERED DOSE INHALER

(71) Applicant: Vitapul Inc., Salt Lake City, UT (US)

(72) Inventors: Teryn Holeman, Salt Lake City, UT (US); Brian Parker, Millcreek, UT (US); Alex Huhn, Salt Lake City, UT (US); Nicholas Wallace, Vineyard, UT (US)

(21) Appl. No.: 17/587,950

(22) Filed: Jan. 28, 2022

Related U.S. Application Data
(60) Provisional application No. 63/142,660, filed on Jan. 28, 2021.

(51) Int. Cl. A61M 15/00

(52) U.S. Cl. CPC A61M 15/0065

(57) ABSTRACT

A small form factor metered dose inhaler is disclosed. The inhaler includes a casing having a longitudinal axis and a transverse axis. The casing has a mouthpiece opening at one end along the longitudinal axis. The casing is configured to house a medicament canister along the longitudinal axis. The medicament canister contains a plurality of doses of a pulmonary medicament and a metered dose valve configured to release a metered dose of the pulmonary medicament through the mouthpiece opening along the longitudinal axis. A mechanical linkage translates an applied input force to the mechanical linkage into an output force along the longitudinal axis configured to actuate the metered dose valve and release the metered dose of the pulmonary medicament in a direction along the longitudinal axis. The aerosolized medicament exits the casing in a fluid flow which is symmetric in a vertical plane along the longitudinal axis.

### CROSS-REFERENCE TO RELATED APPLICATION

This application claims the benefit of U.S. Provisional Application No. 63/142,660, filed Jan. 28, 2021, which application is incorporated herein by reference.

### BACKGROUND

The present invention relates to metered dose inhalers used to deliver rescue medication, a pulmonary medicament, to those with chronic or acute respiratory disease. More specifically, the claimed device has a smaller form factor than the rescue inhalers currently on the market, efficiently releases the rescue medication in a longitudinal direction throughout the inhaler device, and includes a mechanical linkage to translate an applied or input transverse force to an output longitudinal force that actuates a metered dose valve on the medicament canister to release the rescue medication.

In the United States, the incidence of asthma has increased by 40% between 2000 and 2019, and COPD is currently the 3rd leading medical cause of death. Approximately 40 million people are currently diagnosed with asthma or COPD, and an estimated 12 million people have yet to be diagnosed according to the National Institutes of Health. To treat these chronic respiratory diseases, doctors typically prescribe an albuterol rescue inhaler. The rescue inhaler, also known as a metered dose inhaler, is used to deliver metered doses of aerosolized pulmonary medicament to the respiratory tract on an as-needed basis. For example, the standard treatment for bronchopulmonary inflammation and airway narrowing (commonly known as an asthma attack) is to administer three metered doses puffs of albuterol.

More recently, albuterol metered dose inhalers have also been used to treat patients who have acute cases of lower respiratory tract infections, such as the common cold, the flu, and COVID-19. The COVID-19 pandemic has increased the need for albuterol exponentially. The standard inhaler contains 200 metered doses of albuterol and is intended to last a patient for as long as a full year. Patients who are prescribed a metered dose for COVID-19 or other acute illnesses may only need the inhaler for one to two weeks, and it is very unlikely that the patient will use all 200 metered doses. It is unnecessary to prescribe 200 metered doses of albuterol for an acute illness when the patient will throw away the majority of the medication after they have recovered. The practice of prescribing the standard 200 dose inhalers for acute illness has led to unnecessary medical waste and contributed to the FDA's issuance of an active shortage warning for albuterol.

Furthermore, due to the nature of chronic respiratory diseases, patients need access to their inhaler at all times, even though many only use their inhaler a few times per year or less. However, the currently available metered dose inhalers are too large to comfortably carry on your person at all times. They are also too expensive for the patient to purchase multiple inhalers to have one in the car, in their purse, in their office desk, and anywhere else they commonly spend time.

Finally, the L-shaped design of the most common metered dose inhalers creates a complex fluid flow of medicament, air, propellant, and surfactant with asymmetric velocities about the centerline of the mouthpiece. Research has shown that this complex fluid flow causes the direction of the pulmonary medicament leaving the device to dip downward towards the tongue. See, Crosland, B. M., M. R. Johnson, and E. A. Matida, Characterization of the spray velocities from a pressurized metered-dose inhaler. Journal of aerosol medicine and pulmonary drug delivery, 2009. 22(2): p. 85-98. Dunbar, C. A., atomization mechanisms of the pressurized metered dose inhaler. Particulate Science and Technology, 1997. 15(3-4): p. 253-271. Alatrash, A. and E. Matida, Characterization of medication velocity and size distribution from pressurized metered-dose inhalers by phase Doppler anemometry. Journal of Aerosol Medicine and Pulmonary Drug Delivery, 2016. 29(6): p. 501-513. It is hypothesized that this complex fluid flow causes a noticeable amount of the aerosolized medication to land on the tongue of the patient, rather than proceeding straight to the lungs. This known issue results in a less efficient administration of the medication and a weakened treatment of the symptoms.

The metered dose inhaler disclosed herein is not limited to embodiments that solve any disadvantages or that operate only in environments such as those described above. Rather, this background is only provided to illustrate one example technology area where some implementations described herein may be practiced.

### SUMMARY

The present disclosure relates to a metered dose inhaler. In particular, the present disclosure relates to a metered dose inhaler having a small size compared to conventional L-shaped inhalers. The disclosed inhaler uses a smaller medicament canister with fewer metered doses which helps reduce unnecessary medical waste. It also reduces the cost so that multiple inhalers may be economically obtained and makes it more feasible to have multiple inhalers for regularly used locations. The small size increases the practicality of carrying a metered dose inhaler on your person at all times due to the smaller form factor. Finally, the inhaler provides a more efficient and effective administration of the aerosolized medication.

The metered dose inhaler includes a casing having a longitudinal axis and a transverse axis, where the casing may include a mouthpiece opening at one end along the longitudinal axis, where the casing is configured to house a medicament canister along the longitudinal axis, where the medicament canister may include a plurality of doses of a pulmonary medicament and a metered dose valve configured to release a metered dose of the pulmonary medicament through the mouthpiece opening along the longitudinal axis. As described herein, the longitudinal axis passes through a centerline of the mouthpiece when viewing the device from the side. The inhaler also includes a mechanical linkage configured to translate an applied or input force to the mechanical linkage into an output force along the longitudinal axis configured to actuate the metered dose valve and release the metered dose of the pulmonary medicament in a longitudinal direction along the longitudinal axis.

Implementations may include one or more of the following features. The metered dose inhaler comprises a medicament canister. The medicament canister is sized to contain between 20 and 50 doses of the pulmonary medicament. The medicament canister is sized to contain between 30 and 40 doses of the pulmonary medicament. The medicament canister has an outer diameter in the range of 12 mm to 18 mm. The medicament canister has an outer diameter in the range of 14 mm to 16 mm. The metered dose valve has an outer diameter in the range of 12 mm to 18 mm. The metered dose valve has an outer diameter in the range of 14 mm to 16 mm. The medicament canister has a length in the range of 20 mm to 30 mm. The medicament canister has a length in the range of 21 mm to 25 mm. The medicament canister may include an internal dip tube to syphon the pulmonary medicament when the medicament canister is oriented in a horizontal direction. A chamber receives pulmonary medicament released from the metered dose valve in the longitudinal direction and the pulmonary medicament exits the chamber in the longitudinal direction. The casing may include a key ring attachment structure. The metered dose valve may include a stem which is compressed during actuation to release the pulmonary medicament, where the mechanical linkage moves the medicament canister relative to the stem to compress the stem. The mechanical linkage may include a compliant mechanism. The metered dose valve may include a stem which is compressed during actuation to release the pulmonary medicament, where the mechanical linkage moves the stem relative to the medicament canister to compress the stem. The mechanical linkage may include two arms configured to pivot, and where the applied input force to the mechanical linkage causes the two arms to pivot and flex to apply the force along the longitudinal axis to compress the stem. The casing may include one or more air inlet holes configured to allow air to enter the casing. The fluid flow of the pulmonary medicament exiting the casing is symmetric about the centerline of the mouthpiece in a vertical plane along the longitudinal axis. The pulmonary medicament is released from the metered dose valve in the longitudinal direction and exits the casing through the mouthpiece opening in the longitudinal direction. The applied or input force to the mechanical linkage is along the transverse axis.

This summary of the present invention is not intended to describe each illustrated embodiment or every possible implementation of the present invention. The figures and the detailed description that follow, however, do particularly exemplify these embodiments. The metered dose inhaler disclosed herein solves the issues and inconveniencies of currently available metered dose inhalers. Specifically, the disclosed inhaler: helps reduce unnecessary medical waste by using a canister with fewer metered doses; increases the practicality of carrying a metered dose inhaler on your person at all times due to the smaller form factor; makes it more feasible to have multiple inhalers for regularly used locations because of its lower cost; and provides a more efficient and effective administration of the aerosolized medication.

It is to be understood that both the foregoing general description and the following detailed description are exemplary and explanatory and are not restrictive of the invention, as claimed. It should be understood that the various embodiments are not limited to the arrangements and instrumentality shown in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1 is a perspective view of an example embodiment of a metered dose inhaler.

FIG. 2 is a perspective view of an embodiment of a mechanical linkage in the metered dose inhaler of FIG. 1.

FIG. 3 is a cross-sectional representation of a medicament canister.

FIG. 4 is an exploded view of the mechanical linkage shown in FIG. 2.

FIGS. 5A-5C are side views of the mechanical linkage shown in FIG. 2 illustrating the operation of the mechanical linkage as the button is depressed to actuate the metered dose valve and release the pulmonary medicament.

FIGS. 6A-61 show alternative embodiments of mechanical linkages and compliant mechanisms.

FIG. 7 is a perspective view of another example embodiment of a metered dose inhaler.

FIG. 8 is an exploded perspective view of the metered dose inhaler shown in FIG. 7.

FIG. 9 is a cross-section view of the metered dose inhaler shown in FIG. 7.

FIG. 10 is a front view of the metered dose inhaler shown in FIG. 7.

FIG. 11 is a rear view of the metered dose inhaler shown in FIG. 7.

FIGS. 12A-12C are side cross-sectional views of the metered dose inhaler shown in FIG. 7 illustrating the operation of the mechanical linkage as the button is depressed to actuate the metered dose valve and release the pulmonary medicament.

### DETAILED DESCRIPTION OF EMBODIMENTS

The metered dose inhalers, also known as rescue inhalers, that are typically prescribed to patients who have asthma, COPD, or other respiratory diseases or infections, are too large to conveniently carry at all times. Although many patients only use their inhaler a few times per year, it is crucial that the inhaler is readily available when needed, for instance, in the event of an asthma attack. The disclosed invention is a metered dose inhaler that has a smaller form factor than the currently available inhalers, making it more convenient to carry at all times. The smaller form factor also solves other issues in the current art, such as reducing medical waste by using a canister with fewer metered doses; making it more feasible to have multiple inhalers for regularly used locations because of the lower cost of a canister with fewer doses; and providing a more efficient and effective administration of the aerosolized medication.

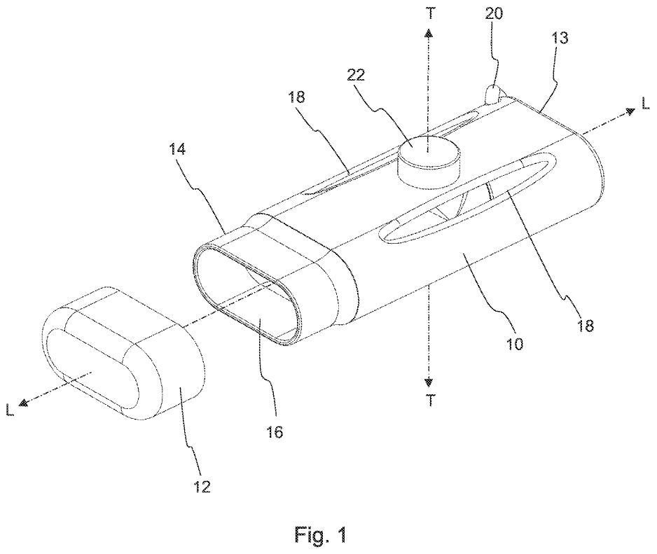

FIG. 1 shows one embodiment of a metered dose inhaler. Metered dose inhaler 1 (“the inhaler”) has a casing 10 and a cap 12. The casing may be manufactured from any suitable material which is durable and light weight. In a disclosed embodiment, casing 10 and cap 12 are constructed of a molded polymeric material or plastic. A plastic construction is advantageous because of the ease and low cost of manufacture, along with the light weight and durability of the material. However, alternative embodiments may utilize other materials, such as aluminum or any other appropriate material.

Non-limiting examples of polymeric materials from which the casing is manufactured include polypropylene, polyethylene, polyvinylchloride, polyurethane, polysulfone, polyphenylsulfone, acrylonitrile butadiene styrene, polyethylene terephthalate, polyamide, polyarcrylates, polyvinyl acetates, polyimide, polyamideimide, polymethylmethacrylate, polyetherimide, polyetheretherketone, polyethersulfone, polycarbonate, and polyester.

Casing 10 has a generally obround cross section when viewed down its length or longitudinal axis L. The casing 10 is hollow. End 13 of casing 10 is a closed end and end 16 is open. A transverse axis T is also shown.

Mouthpiece 14 is situated at end 16. The mouthpiece 14 may have a similar cross section to casing 10. The mouthpiece 14 may have a different cross section compared to the casing 10. In the embodiment shown in FIG. 1, the cross-sectional area of mouthpiece 14 is smaller than the cross-sectional area of casing 10. The smaller cross-sectional area of mouthpiece 14 allows cap 12 to fit over and slide over both end 16 and mouthpiece 14. When using the inhaler, a user will place their mouth over mouthpiece 14 and inhale through their mouth.

Cap 12 is configured to slide over end 16 and mouthpiece 14 and remain there when the inhaler is not in use. Cap 12 is further configured to be slid off mouthpiece 14 and end 16 prior to using the inhaler. In the disclosed embodiment, cap 12 remains slid over end 16 and mouthpiece 14 when the inhaler is not in use by virtue of a friction fit between the mouthpiece and cap. Alternate embodiments of the disclosed invention may employ the use of detents or other suitable retention means to keep cap 12 in place when the inhaler is not in use. The purpose of cap 12 is to prevent foreign objects from entering casing 10 through end 16, as well as to keep mouthpiece 14 clean and free of foreign objects. Alternative embodiments may use a pivoting or hinged cap configuration rather than a sliding cap configuration to cover end 16 and mouthpiece 14.

The casing 10 may have a key ring attachment structure to facilitate attachment of a key ring. The attachment structure may be a hole or opening. It may be one or more resilient structures to which a key ring may be attached. As shown in FIG. 1, key ring 20 is situated near end 13 and is used to securely attach the inhaler to a keychain, lanyard, or other tether. A key ring attachment structure or key ring 20 may alternatively be positioned near end 16 or on cap 12.

Air inlets 18 are situated on the top side of casing 10. The purpose of air inlets 18 is to promote an efficient flow of air through casing 10 when a user places their mouth over mouthpiece 14 and inhales so that the fluid flow is symmetric in a vertical plane as it exits the mouthpiece. The vertical plane may comprise the longitudinal axis. The vertical plane may comprise the longitudinal and transverse axes. The disclosed inhaler is configured to cause the pulmonary medicament to exit the casing in fluid flow which is symmetric about the centerline of the mouthpiece in a vertical plane along the longitudinal axis.

It is to be understood that the direction of the pulmonary medicament fluid flow as it exits the mouthpiece is a general direction. Because of turbulence, individual particulates of pulmonary medicament may be expected to spread, disperse, and otherwise deviate from the longitudinal axis. Thus, as used herein, the fluid flow direction is a general direction of the spray as opposed to each individual streamline or particle of the spray. Thus, the flow of the pulmonary medicament as it exits the metered dose valve of the medicament canister is in a generally linear direction. See, Crosland, B. M., M. R. Johnson, and E. A. Matida, Characterization of the spray velocities from a pressurized metered-dose inhaler. Journal of aerosol medicine and pulmonary drug delivery, 2009. 22(2): p. 85-98. Dunbar, C. A., atomization mechanisms of the pressurized metered dose inhaler. Particulate Science and Technology, 1997. 15(3-4): p. 253-271. Alatrash, A. and E. Matida, Characterization of medication velocity and size distribution from pressurized metered-dose inhalers by phase Doppler anemometry. Journal of Aerosol Medicine and Pulmonary Drug Delivery, 2016. 29(6): p. 501-513. The fluid flow direction of the pulmonary medicament is also mirrored about the centerline of the mouthpiece in a vertical plane along the same longitudinal axis. As a result, the exit velocity and pressure fields are the same on the top and bottom of the mouthpiece. The fluid flow direction of the pulmonary medicament is steady with time. Without being bound by theory, it is believed the linear fluid flow direction of the pulmonary medicament along the longitudinal axis results in a more direct and effective dose of medication to the user's lungs, rather than the medication landing on the user's tongue.

Air inlets 18 may alternatively be situated on end 13, on the bottom side of casing 10, or on the sides of casing 10 and may include fewer or more air inlets. Additionally, the air inlets may be a different shape than what is shown in FIG. 1. Sufficient air inlets are provided to enable the symmetric, planar air velocity at the mouthpiece or exit of the inhaler as described herein. The air inlets are preferably located sufficiently far from the mouthpiece so that the air has enough distance and time to mix and normalize in the casing to enforce the described flow characteristics before leaving the inhaler.

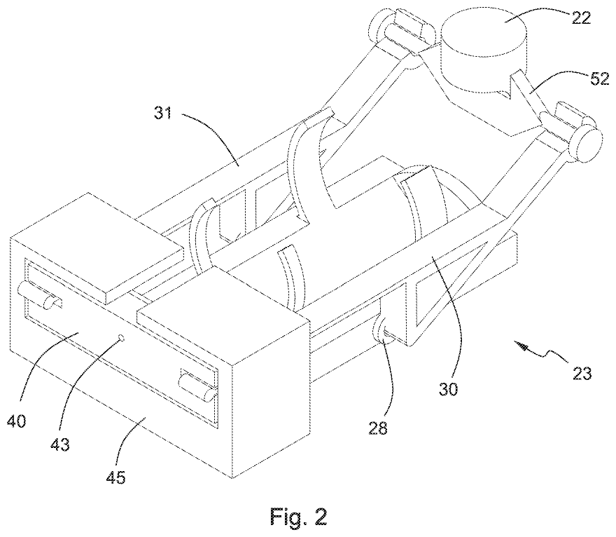

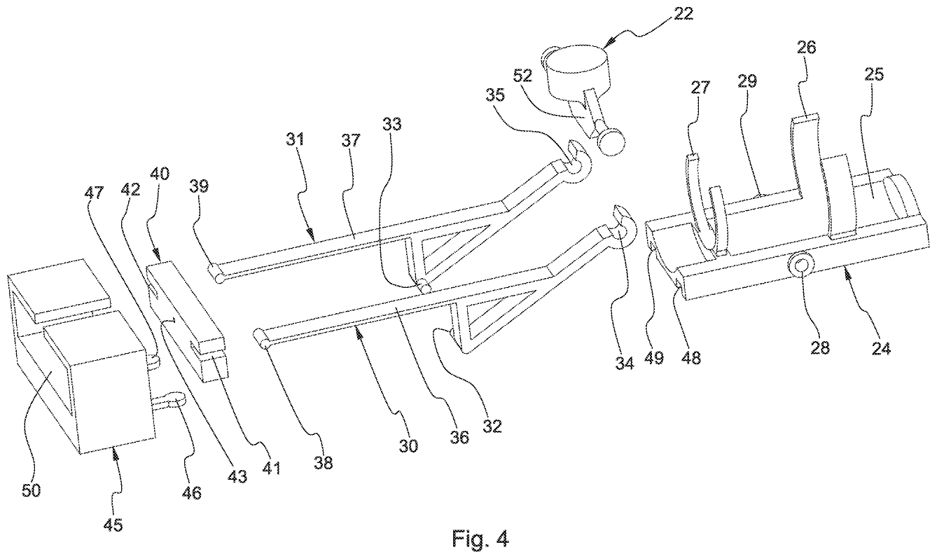

FIG. 2 is a drawing of one possible mechanical linkage that may be situated inside the hollow cavity of casing 10. Mechanical linkage 23 is friction fit into casing 10 and is comprised of six separate parts, all of which are constructed of plastic. Plastic is advantageous because it is both flexible and durable. The six separate parts comprising mechanical linkage 23 are best seen in FIG. 4 and include a canister housing 24, left lever arm 30, right lever arm 31, button 22, slider block 40, and slider housing 45. Mechanical linkage 23 is oriented within casing 10 such that slider housing 45 is near end 16.

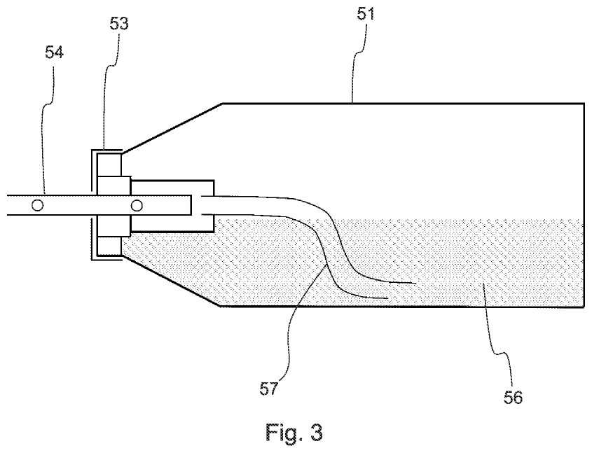

Canister housing 24 is configured to hold a medicament canister with a metered dose valve. FIG. 3 illustrates a medicament canister 51 having a metered dose valve 53 and stem 53. The medicament canister 53 contains a pulmonary medicament 56, such as albuterol sulfate, and an inhalation aerosol, such as hydrofluoroalkanes (HFAs). The medicament canister 51 may comprise an internal dip tube 57 to syphon the pulmonary medicament 56 when the medicament canister is oriented in a horizontal direction.

Known medicament canisters contain a given number of doses per unit. The standard number of doses ranges from 60 to 200 doses. An important feature of the disclosed invention is the use of a much smaller size medicament canister containing fewer doses than typical. The smaller size enables the inhaler to be manufactured with a smaller form factor. A medicament canister with fewer doses eliminates unnecessary medical waste common with standard 200-dose medicament canisters.

The medicament canister outer diameter may range from about 12 mm to about 18 mm, e.g., about 12, 13, 14, 15, 16, 17, or 18 mm, where any of the stated values can form an upper or lower endpoint of a range.

The medicament canister length may range from about 15 mm to about 30 mm, e.g., about 15, 16, 17, 18, 19, 20, 21, 22, 23, 24, 25, 26, 27, 28, 29, or 30 mm, where any of the stated values can form an upper or lower endpoint of a range.

The metered dose valve outer diameter may range from about 12 mm to about 18 mm, e.g., about 12, 13, 14, 15, 16, 17, or 18 mm, where any of the stated values can form an upper or lower endpoint of a range.

The medicament canister may contain from about 20 to about 50 doses of the pulmonary medicament, e.g., about 20, 25, 30, 35, 40, 45, or 50 doses, where any of the stated values can form an upper or lower endpoint of a range.

The canister rests horizontally in curved trough 25 and is held in place by both main clamping arms 26 and secondary clamping arms 27. The main clamping arms and the secondary clamping arms are configured to prevent the canister from moving either vertically or horizontally. The canister housing is configured to hold the canister so that the valve end of the canister faces towards slider block 40.

Canister housing 24 has two mounting holes, left arm mounting hole 28 and right arm mounting hole 29. The two mounting holes and are configured to receive left pin 32 on left lever arm 30 and right pin 33 on right lever arm 31, respectively. When the respective pin is inserted into the respective mounting hole, the respective lever arm is attached to cannister housing 24. It should be understood that various separate parts disclosed herein may be combined into one injection molded part. Similarly, instead of having the rotational hinges shown in 32 and 33, it is within the scope of the disclosed invention to replace these structures with compliant hinges.

Button 22 is attached to trunnion 52, which rests on left trunnion mount 34 of left lever arm 30 and right trunnion mount 35 of right lever arm 31. When a downward force is applied to button 22, left lever arm 30 and right lever arm 31 pivot about left pin 32 and right pin 33, respectively.

Left lever arm 30 and right lever arm 31 each include a flexible member, left flexible member 36 and right flexible member 37, respectively. Left flexible member 36 has left bulb 38 disposed on its end and right flexible member 37 has right bulb 39 on its end. The two bulbs interface with left and right slots 41 and 42 on slider block 40. Slider block 40 and left and right slots 41 and 42 are configured to interface with the flexible members, such that the flexible members are prevented from moving vertically, as well as prevented from moving in a horizontal plane independently from slider block 40.

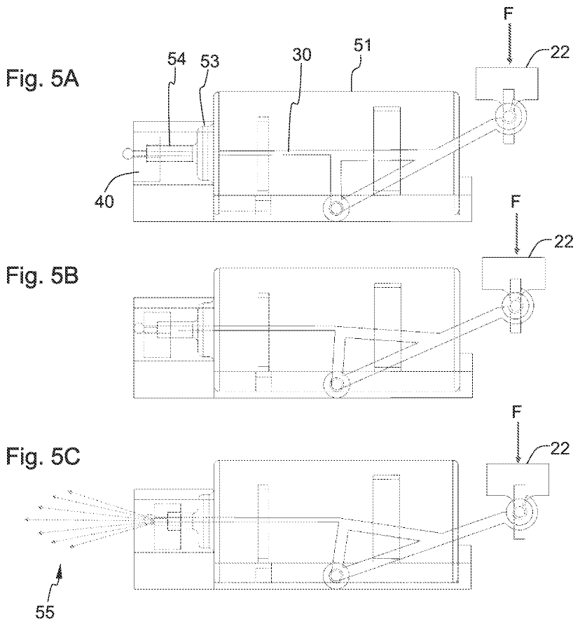

As shown in FIGS. 5A-5C, when a downward force F is applied to button 22, the lever arms 30, 31 pivot about their respective pins, 32, 33. The ends of flexible members 36 and 37 are unable to move in a vertical direction and pivot with the rest of the lever arm because of the interaction with slider block 40. As a result, the flexible members flex upwards, thereby applying a horizontal force on slider block 40. This force causes slider block 40 to slide towards the canister housing.

Slider block 40 includes spray nozzle hole 43, which is configured to receive a spray nozzle on a medicament canister 51 with a metered dose valve 53. The metered dose valve has a stem 54 which is actuated by compressing the stem of the spray nozzle in towards the canister, thereby releasing the metered dose of aerosolized medicament 55. When slider block 40 slides towards the canister housing, it actuates the canister's metered dose valve, which results in the aerosolized medication passing through spray nozzle hole 43. Because the spray nozzle hole is near end 16, the aerosolized medicament passes through mouthpiece 14 when the user places their mouth on the mouthpiece and inhales.

Slider housing 45 receives slider block 40 in opening 50 and is configured to allow and ensure slider block 40 slides normal to the canister. Additionally, the slider housing 45 prevents slider block 40 from moving vertically. Slider housing 45 further includes tabs 46 and 47 that interface with slots 48 and 49 on canister housing 24, respectively, to prevent the components of mechanical linkage 23 from separating or moving independently. It should be understood that some of these separate parts may be combined into one injection molded part.

The various components of the metered dose inhaler may be constructed generally out of any materials known to be suitable in the art.

It will be appreciated that the mechanical linkage shown in FIGS. 2 and 4 is only one possible way of converting an applied input force in one direction, such as a transverse or vertical direction, into an output force in another direction, such as a longitudinal or horizontal direction.

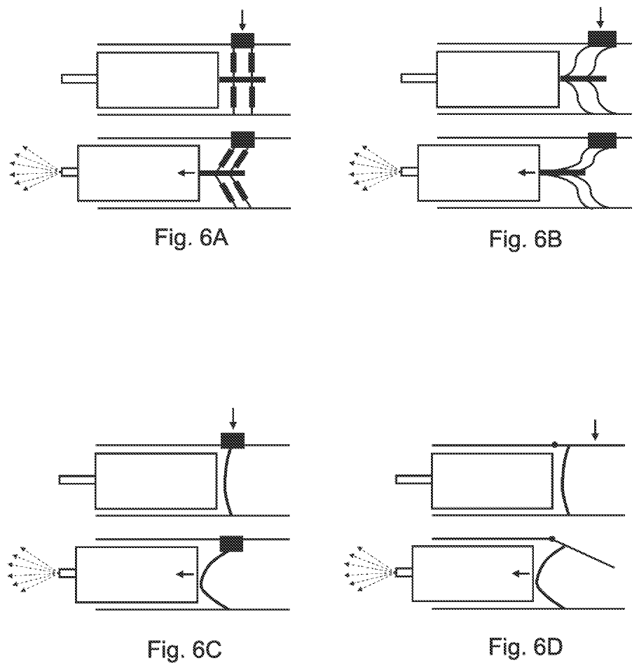

FIGS. 6A-61 illustrate a variety of possible mechanical linkages and compliant mechanisms which one might utilize in the metered dose inhaler disclosed herein. A compliant mechanism is a flexible mechanism that achieve force and motion transmission through elastic body deformation. It will be appreciated that numerous possible mechanical linkages and compliant mechanisms may be implemented, and the invention is not limited to any specific mechanical linkage or compliant mechanism disclosed herein. As used herein, the term “mechanical linkage” includes compliant mechanisms which transmits an applied input force in one direct into an output force in a different direction. For example, the mechanical linkage may transmit an applied input force in a transverse direction into an output force in the longitudinal direction.

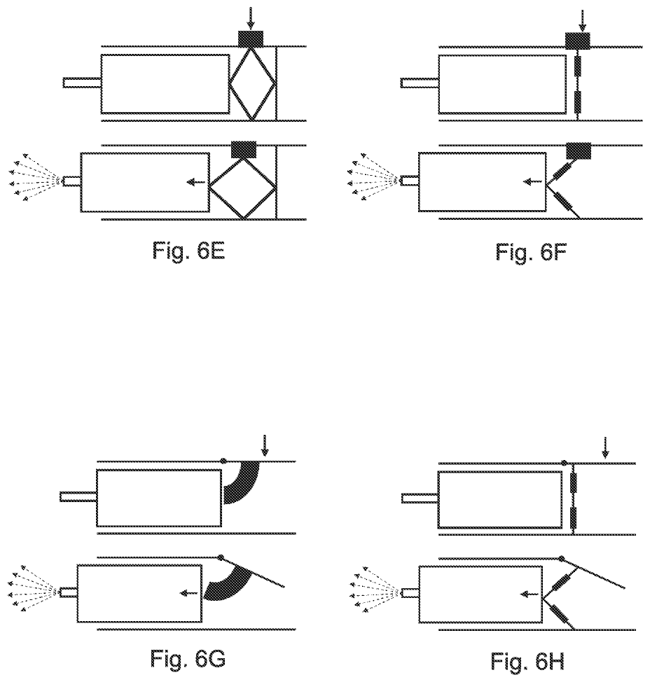

FIGS. 7-12C illustrate an embodiment of another metered dose inhaler 60. Metered dose inhaler 60 (“the inhaler”) has a casing 62. Like casing 10, the casing 62 may be manufactured from any suitable material which is durable and light weight. In a disclosed embodiment, the casing 62 may be constructed of a molded polymeric material or plastic. A plastic construction is advantageous because of the ease and low cost of manufacture, along with the light weight and durability of the material. However, alternative embodiments may utilize other materials, such as aluminum or any other appropriate material.

Non-limiting examples of polymeric materials from which the casing 62 is manufactured include polypropylene, polyethylene, polyvinylchloride, polyurethane, polysulfone, polyphenylsulfone, acrylonitrile butadiene styrene, polyethylene terephthalate, polyamide, polyarcrylates, polyvinyl acetates, polyimide, polyamideimide, polymethylmethacrylate, polyetherimide, polyetheretherketone, polyethersulfone, polycarbonate, and polyester.

Casing 62 has a generally obround cross section when viewed down its length or longitudinal axis L. The casing 62 is hollow. A transverse axis T is also shown.

A mouthpiece 64 is situated at end 66. When using the inhaler 60, a user will place their mouth over mouthpiece 64 and inhale through their mouth.

A locking cap 68 is disposed at end 70 of casing 62. The locking cap includes a depressible button 72.

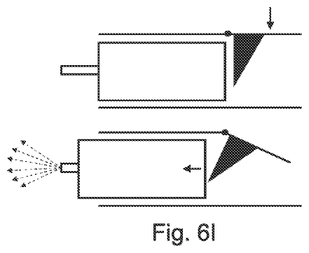

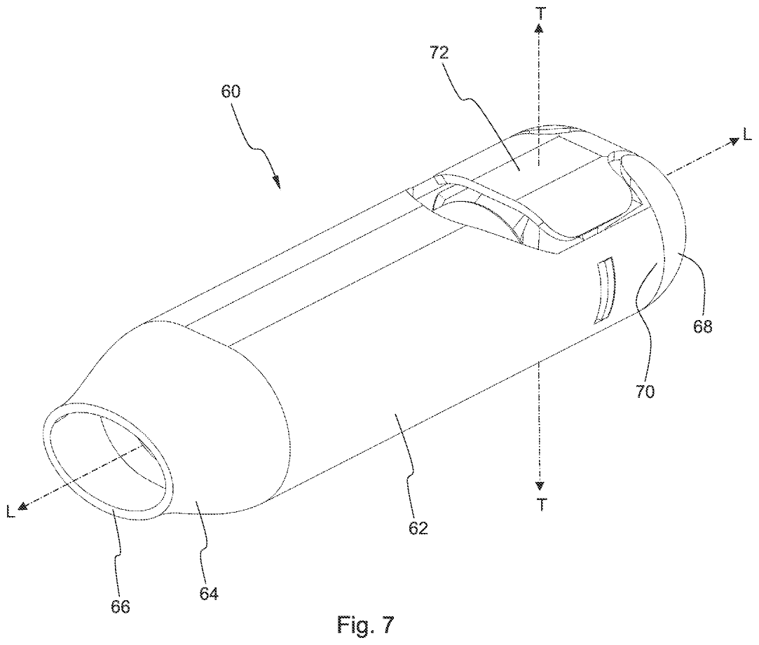

As shown in FIGS. 8 and 9, the locking cap 68 is configured to retain a mechanical linkage 74. The locking cap may include a fastener, such as a pair of elastic hooks 76 which are received in openings 78 formed in casing 62. Other known and novel fastening structures may be used to secure the locking cap to the casing.

The casing 62 includes an interior space 80 sized and configured to house a medicament canister along the longitudinal axis L. As disclosed above, the medicament canister may include a plurality of doses of a pulmonary medicament and a metered dose valve configured to release a metered dose of the pulmonary medicament through the mouthpiece 64 along the longitudinal axis L. The metered dose valve has a stem which is actuated by compressing the stem of the spray nozzle in towards the canister, thereby releasing the metered dose of aerosolized medicament. As shown in FIG. 9, the casing 62 includes a wall 82 adjacent the mouthpiece 64. The wall 82 includes a stem receiver 84 which is sized and configured to receive the stem of a metered dose valve of a pulmonary medicament canister. The wall 82 includes a chamber 85 to receive pulmonary medicament released from the metered dose valve through the stem in the longitudinal direction. The wall 82 further includes a spray nozzle hole 86. Pulmonary medicament exits the chamber 85 in the longitudinal direction through the spray nozzle hole 86, which allows aerosolized pulmonary medicament to pass into the mouthpiece.

The mechanical linkage 74 is fabricated of an elastic material. The mechanical linkage 74 transmits an applied force A to the button 72 into an output force B in a different direction, such as the longitudinal axis.

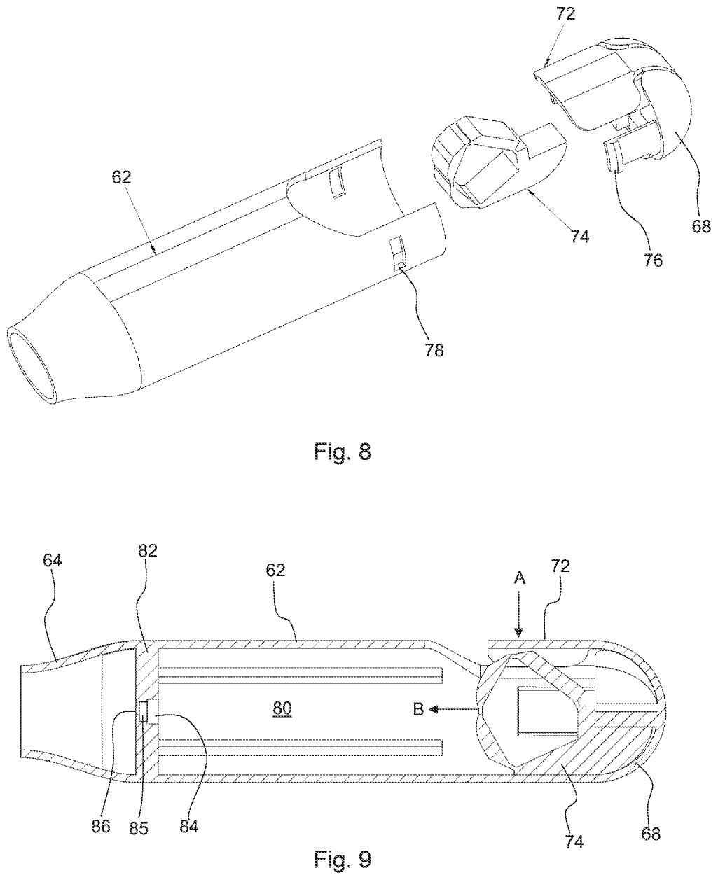

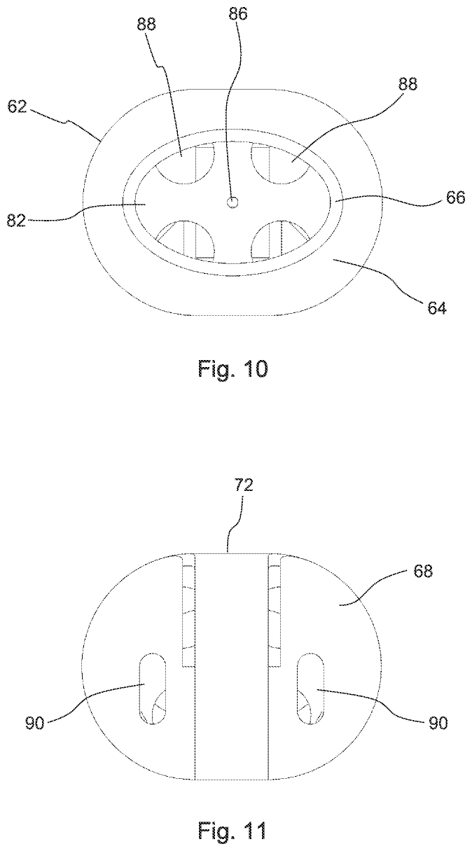

As shown in FIG. 10, air inlets 88 are formed in wall 82 and allow air to pass through when a user places their mouth over mouthpiece 64 and inhales. As shown in FIG. 11, air inlets 90 are formed in locking cap 68 and allow air to pass through when a user places their mouth over mouthpiece 64 and inhales.

The air inlets 88, 90 promote an efficient flow of air through casing 62 when a user places their mouth over mouthpiece 64 and inhales so that the fluid flow is symmetric about the centerline of the mouthpiece in a vertical plane along the longitudinal axis as it exits the mouthpiece 64. The vertical plane may comprise the longitudinal axis. The vertical plane may comprise the longitudinal and transverse axes.

It is to be understood that the direction of the pulmonary medicament fluid flow as it exits the mouthpiece is a general direction. Because of turbulence, individual particulates of pulmonary medicament may be expected to spread, disperse, and otherwise deviate from the longitudinal axis. Thus, as used herein, the fluid flow direction is a general direction of the spray as opposed to each individual streamline or particle of the spray. Thus, the flow of the pulmonary medicament as it exits the metered dose valve of the medicament canister is in a generally linear direction. The fluid flow direction of the pulmonary medicament is also mirrored about the centerline of the mouthpiece in a vertical plane along the same longitudinal axis. As a result, the exit velocity and pressure are the same on the top and bottom of the mouthpiece. The fluid flow direction of the pulmonary medicament is steady with time. Without being bound by theory, it is believed the linear fluid flow direction of the pulmonary medicament along the longitudinal axis results in a more direct and effective dose of medication to the user's lungs, rather than the medication landing on the user's tongue.

Air inlets 88, 90 may alternatively be situated one or more sides of casing 62 and may include fewer or more air inlets. Additionally, the air inlets may be a different shape than what is shown in FIGS. 10 and 11. Sufficient air inlets are provided to enable a symmetric, planar air velocity at the mouthpiece 64 or exit of the inhaler as described herein. The air inlets are preferably located sufficiently far from the mouthpiece so that the air has enough distance and time to mix and normalize in the casing to provide the described flow characteristics before leaving the inhaler.

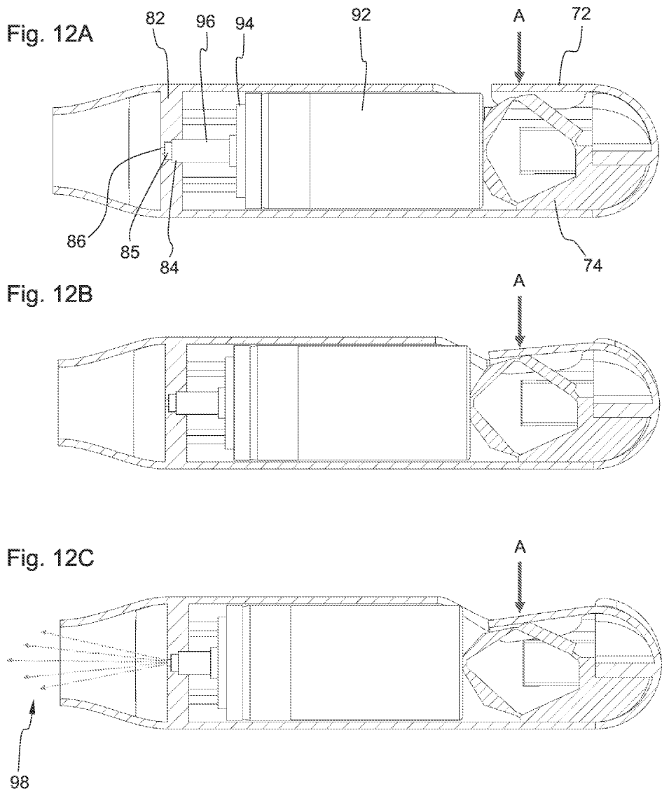

FIGS. 12A-12C illustrate the action of the inhaler when a downward force A is applied to button 72. As the button 72 is pressed with downward force A, the mechanical linkage 74 is compressed in a manner than creates the force B, shown in FIG. 9. This horizontal force B moves medicament canister 92 in a longitudinal direction towards wall 82. The medicament canister 92 includes a metered dose valve 94. The metered dose valve has a stem 96. The stem is positioned in the stem receiver 84. The stem 96 is actuated by compressing the stem of the spray nozzle in towards the medicament canister, thereby releasing the metered dose of aerosolized medicament 98.

Because the spray nozzle hole 86 is near end 66, the aerosolized medicament 98 passes through mouthpiece 64 when the user places their mouth on the mouthpiece and inhales. The aerosolized medicament 98 exits the casing 62 in a fluid flow which is symmetric in a vertical plane. In some embodiments, the aerosolized medicament 98 exits the casing 62 in a fluid flow which is symmetric in a vertical plane along the longitudinal axis L.

### Embodiments

Various embodiments are listed below. It will be understood that the embodiments listed below may be combined with all aspects and other embodiments in accordance with the scope of the invention.

Embodiment 1. A metered dose inhaler comprising: a casing having a longitudinal axis and a transverse axis, wherein the casing comprises a mouthpiece opening at one end along the longitudinal axis, wherein the casing is configured to house a medicament canister along the longitudinal axis, wherein the medicament canister comprises a plurality of doses of a pulmonary medicament and a metered dose valve configured to release a metered dose of the pulmonary medicament through the mouthpiece opening along the longitudinal axis; and a mechanical linkage configured to translate an applied force to the mechanical linkage into a force along the longitudinal axis configured to actuate the metered dose valve and release the metered dose of the pulmonary medicament in a longitudinal direction along the longitudinal axis.

Embodiment 2. The metered dose inhaler of embodiment 1, wherein the medicament canister is sized to contain between 20 and 50 doses of the pulmonary medicament

Embodiment 3. The metered dose inhaler of any preceding embodiment, wherein the medicament canister is sized to contain between 30 and 40 doses of the pulmonary medicament.

Embodiment 4. The metered dose inhaler of any preceding embodiment, wherein the medicament canister has an outer diameter in the range of 12 mm to 18 mm.

Embodiment 5. The metered dose inhaler of any preceding embodiment, wherein the medicament canister has an outer diameter in the range of 14 mm to 16 mm.

Embodiment 6. The metered dose inhaler of any preceding embodiment, wherein the metered dose valve has an outer diameter in the range of 12 mm to 18 mm.

Embodiment 7. The metered dose inhaler of any preceding embodiment, wherein the metered dose valve has an outer diameter in the range of 14 mm to 16 mm.

Embodiment 8. The metered dose inhaler of any preceding embodiment, wherein the medicament canister has a length in the range of 15 mm to 30 mm.

Embodiment 9. The metered dose inhaler of any preceding embodiment, wherein the medicament canister has a length in the range of 19 mm to 24 mm.

Embodiment 10. The metered dose inhaler of any preceding embodiment, wherein the medicament canister comprises an internal dip tube to syphon the pulmonary medicament when the medicament canister is oriented in a horizontal direction.

Embodiment 11. The metered dose inhaler of any preceding embodiment, further comprising a chamber connected to the metered dose valve to permit mixing and expansion of the pulmonary medicament as it is released from the metered dose valve, wherein the chamber receives pulmonary medicament released from the metered dose valve in the longitudinal direction and the pulmonary medicament exits the chamber in the longitudinal direction.

Embodiment 12. The metered dose inhaler of any preceding embodiment, wherein the casing comprises a key ring attachment structure.

Embodiment 13. The metered dose inhaler of any preceding embodiment, wherein the metered dose valve comprises a stem which is compressed during actuation to release the pulmonary medicament, wherein the mechanical linkage moves the medicament canister relative to the stem to compress the stem.

Embodiment 14. The metered dose inhaler of any embodiment 1 through 12, wherein the metered dose valve comprises a stem which is compressed during actuation to release the pulmonary medicament, wherein the mechanical linkage moves the stem relative to the medicament canister to compress the stem.

Embodiment 15. The metered dose inhaler of any preceding embodiment, wherein the mechanical linkage comprises a compliant mechanism.

Embodiment 16. The metered dose inhaler of any preceding embodiment, wherein the mechanical linkage comprises two arms configured to pivot, and wherein the applied force to the mechanical linkage causes the two arms to pivot and flex to apply the force along the longitudinal axis to compress the stem.

Embodiment 17. The metered dose inhaler of any preceding embodiment, wherein the casing comprises one or more air inlet holes configured to allow air to enter the casing.

Embodiment 18. The metered dose inhaler of any preceding embodiment, wherein the pulmonary medicament exits the casing in a fluid flow which is symmetric in a vertical plane along the longitudinal axis.

Embodiment 19. The metered dose inhaler of any preceding embodiment, wherein the pulmonary medicament exits the casing in a fluid flow which is symmetric in a vertical plane.

Embodiment 20. The metered dose inhaler of any preceding embodiment, wherein the main direction of the pulmonary medicament fluid flow exiting the casing is constant in time.

Embodiment 21. The metered dose inhaler of any preceding embodiment, wherein the pulmonary medicament is released from the metered dose valve in the longitudinal direction and exits the casing through the mouthpiece opening in the longitudinal direction.

Embodiment 22. The metered dose inhaler of any preceding embodiment, wherein the applied force to the mechanical linkage is along the transverse axis.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments. Although specific terms are employed herein, they are used in a generic and descriptive sense only, and not for purposes of limitation.

While the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A metered dose inhaler comprising:
a casing having a longitudinal axis and a transverse axis, wherein the casing comprises a mouthpiece opening at one end along the longitudinal axis, wherein the casing is configured to house a medicament canister along the longitudinal axis, wherein the medicament canister comprises a plurality of doses of a pulmonary medicament and a metered dose valve configured to release a metered dose of the pulmonary medicament through the mouthpiece opening along the longitudinal axis; and
a mechanical linkage configured to translate an applied force to the mechanical linkage into a force along the longitudinal axis configured to actuate the metered dose valve and release the metered dose of the pulmonary medicament in a longitudinal direction along the longitudinal axis.

2. The metered dose inhaler of claim 1, wherein the medicament canister is sized to contain between 20 and 50 doses of the pulmonary medicament.

3. The metered dose inhaler of claim 1, wherein the medicament canister is sized to contain between 30 and 40 doses of the pulmonary medicament.

4. The metered dose inhaler of claim 1, wherein the medicament canister has an outer diameter in the range of 12 mm to 18 mm.

5. The metered dose inhaler of claim 1, wherein the medicament canister has an outer diameter in the range of 14 mm to 16 mm.

6. The metered dose inhaler of claim 1, wherein the metered dose valve has an outer diameter in the range of 12 mm to 18 mm.

7. The metered dose inhaler of claim 1, wherein the metered dose valve has an outer diameter in the range of 14 mm to 16 mm.

8. The metered dose inhaler of claim 1, wherein the medicament canister has a length in the range of 15 mm to 30 mm.

9. The metered dose inhaler of claim 1, wherein the medicament canister has a length in the range of 19 mm to 24 mm.

10. The metered dose inhaler of claim 1, wherein the medicament canister comprises an internal dip tube to syphon the pulmonary medicament when the medicament canister is oriented in a horizontal direction.

11. The metered dose inhaler of claim 1, further comprising a chamber connected to the metered dose valve to permit mixing and expansion of the pulmonary medicament as it is released from the metered dose valve, wherein the chamber receives pulmonary medicament released from the metered dose valve in the longitudinal direction and the pulmonary medicament exits the chamber in the longitudinal direction.

12. The metered dose inhaler of claim 1, wherein the casing comprises a key ring attachment structure.

13. The metered dose inhaler of claim 1, wherein the metered dose valve comprises a stem which is compressed during actuation to release the pulmonary medicament, wherein the mechanical linkage moves the medicament canister relative to the stem to compress the stem.

14. The metered dose inhaler of claim 1, wherein the mechanical linkage comprises a compliant mechanism.

15. The metered dose inhaler of claim 1, wherein the metered dose valve comprises a stem which is compressed during actuation to release the pulmonary medicament, wherein the mechanical linkage moves the stem relative to the medicament canister to compress the stem.

16. The metered dose inhaler of claim 1, wherein the mechanical linkage comprises two arms configured to pivot, and wherein the applied force to the mechanical linkage causes the two arms to pivot and flex to apply the force along the longitudinal axis to compress the stem.

17. The metered dose inhaler of claim 1, wherein the casing comprises one or more air inlet holes configured to allow air to enter the casing.

18. The metered dose inhaler of claim 1, wherein the pulmonary medicament exits the casing in a fluid flow which is symmetric in a vertical plane along the longitudinal axis.

19. The metered dose inhaler of claim 1, wherein the pulmonary medicament exits the casing in a fluid flow which is symmetric in a vertical plane.

20. The metered dose inhaler of claim 1, wherein the main direction of the pulmonary medicament fluid flow exiting the casing is constant in time.

21. The metered dose inhaler of claim 1, wherein the pulmonary medicament is released from the metered dose valve in the longitudinal direction and exits the casing through the mouthpiece opening in the longitudinal direction.

22. The metered dose inhaler of claim 1, wherein the applied force to the mechanical linkage is along the transverse axis.
